(19) United States
(12) Patent Application Publication
(10) Pub. No.: US 2024/0038383 A1
(43) Pub. Date: Feb. 1, 2024

(54) Health Monitoring System

(71) Applicant: Innsightful, Inc., Ann Arbor, MI (US)

(72) Inventors: Farzad Ehsani, Sunnyvale, CA (US); Demitrios L. Master, Cupertino, CA (US)

(21) Appl. No.: 18/039,751

(22) PCT Filed: Nov. 30, 2021
(86) PCT No.: PCT/US2021/061137

§ 371 Date: Jun. 1, 2023

Related U.S. Application Data
(60) Provisional application No. 63/120,255, filed on Dec. 2, 2020.

(51) Int. Cl. G16H 40/67; G16H 50/20

(52) U.S. Cl. CPC G16H 40/67; G16H 50/20

(57) ABSTRACT

A system for health monitoring comprised of one or more neural networks. A first function of the one or more neural networks includes an autoencoder functionality, tuned to have high recall, configured for monitoring data and for detecting an anomaly within the data. A second function of the one or more neural networks is a false positive reduction (FPR) function, configured for distinguishing false positive anomalies from true positive anomalies by analyzing the data or querying an individual. Confirmed positive anomalies are classified by a classification engine. The system may be configured to train one or more machine learning models thereof, such that the system is trained for monitoring of the individual.

### TECHNICAL FIELD

The present invention relates to an artificial intelligence (AI) system and method of use for monitoring health of an individual. More particularly, the system detects and classifies higher-order patterns based on data which corresponds to one or more biological signals of the individual for initial detection or continued management of a condition, disease, or disorder by evaluating a plurality of inputs to determine whether the individual is symptomatic or asymptomatic for the condition, disease, or disorder and includes an autoencoder functionality configured for anomaly detection with high recall of potentially positive anomalies, which are passed on to a false positive reduction (FPR) functionality for elimination of false positive anomalies where confirmed positive anomalies are classified by a classification engine and reported to higher-order logic for additional processing and improved health monitoring.

### BACKGROUND

Many serious health conditions go unnoticed because of their subtle presentation, gradual onset, or similarity with benign conditions which require little or no intervention. The risks associated with not treating some conditions are significant. If an individual exhibits signs or symptoms of a particular condition, disease, or disorder, then the individual would need to be aware of this to take appropriate precautions or other actions for treatment or management. Unfortunately, many individuals may not have access to in-person healthcare treatment for a variety of reasons. As a result, these individuals may not receive a diagnosis for a serious condition such as a heart condition, diabetes, cancer, or a communicable disease such as a viral infection, such as the common cold or even COVID-19.

As of Nov. 22, 2021, there were over 47.7 Million people in the US and over 258 million worldwide known to be infected with COVID-19, with number of cases still increasing. As of Nov. 22, 2021, over 770K people have died in the US alone, and over 5.15 Million people have died worldwide. The worldwide COVID-19 fatality rate is currently estimated at 2.4%, while for the elderly (60+) it is estimated at 6%. Early detection and treatment (whether isolation, hospitalization, etc.) is a key for this and future pandemics.

More specifically, COVID-19 is a respiratory tract infection with a spectrum of clinical symptoms that varies from asymptomatic or mild forms to moderate and severe disease. Symptoms include fever (>37.5° C.), dry cough, fatigue, myalgia, sore throat, nasal congestion, shortness of breath (dyspnea), acute respiratory distress syndrome (ARDS) and, rarely diarrhea, nausea and vomiting. As the specificity of these symptoms creates challenges, a more specific sign of the viral infection is the development of an interstitial pneumonia, often bilateral, in a large proportion of cases. Even more challenging is continuing to provide care and monitoring for those impact that also have underlying chronic conditions such as heart disease, diabetes, COPD, or hypertension. Since COVID-19 is 25-30 times more deadly for these individuals, they are at even greater risk compared to the general population.

The large number of mild, suspected infected and even asymptomatic patients suggests that many of these patients can be managed at home or in non-health facilities to limit disease spread. To monitor and manage patient care under these circumstances, care providers must rely on the patient proactively communicating with them about symptoms and disease progression. This may not always occur, and when it does occur, it may overload the caretaker because the patient-to-caretaker ratio is much higher than ordinarily encountered, and caretakers may be unable to effectively allocate their time and resources to those most in need if they are performing triage, managing communications, prioritizing patients, and delivering care. Further, patients may lack the ability to effectively communicate about their condition.

Optimally, care providers would be able to monitor clinical parameters and acquire epidemiological, anamnestic and clinicopathological data to safeguard health via a remote active monitoring by physicians. As remote medicine continues to develop, these approaches could be implemented for population screening and to make a triage at distance, reducing the burdening of healthcare structures with positive effects on pandemic containment and patients' care. Relevant biological signals that can be measured remotely include heart rate (HR) and heart rate variability (HRV), blood pressure (BP), electrodermal activity (EDA), oxygen saturation (SpO2), physical motion, body temperature, and respiratory rate. However, currently, there is no effective way of passively detecting viral infection such as COVID-19, emerging strains or second waves, or even the severe seasonal flu seasons.

Accordingly, there exists a need for passive, remote monitoring technology with automated dialog verification for a variety of conditions, diseases, and disorders, including rapidly emerging infection diseases and chronic disease-related conditions. The present system and shown, described and claimed herein addresses this unmet need.

### SUMMARY

The present system provides an end-to-end platform to monitor a variety of health conditions via a biosensor device and a software application. The system can be utilized by healthcare providers for diagnosis, non-contact therapeutics, monitoring, and population management. The system may be used for pandemic and non-pandemic scenarios and may be easily scaled according to need. In embodiments, the system may be used to monitor, identify, and alleviate mental health issues, and may be utilized to monitor at-risk patients and those who have received a diagnosis and require remote or at-home monitoring. In embodiments, the system includes a biosensor device, a biological signal anomaly detection artificial intelligence (AI) engine, an automated conversation agent, real-time connectivity with health care providers, and a regulatory-compliant (e.g., HIPAA-compliant) backend.

A system having a biosensor device used by a user, the system having at least one hardware processor and at least one machine-readable media for storing instructions that cause the at least one hardware processor to perform operations for health monitoring when executed by the one or more hardware processors, the operations including the steps of 1) monitoring data generated by the biosensor device, the data corresponding to one or more biological properties of an individual; 2) detecting an anomaly of the data; and 3) determining whether the anomaly is a false positive anomaly or a true positive anomaly by querying the user and/or analyzing the data.

In some embodiments, the instructions have one or more neural networks comprising an autoencoder functionality tuned to have high recall, and the autoencoder configured to perform the monitoring of the data and the detecting of the anomaly of the data. In other embodiments, one or more neural networks includes a false positive reduction (FPR) functionality configured to perform the determining whether the anomaly is the false positive anomaly or the true positive anomaly. Further, the one or more neural networks may include a classification engine functionality configured to perform a classifying of the anomaly and to perform a reporting of the anomaly to a pre-determined logic of the instructions. The operations may further include training one or more machine learning (ML) models on the anomaly.

The one or more biological properties may include a biological property selected from a group consisting of: a heart rate (EIR), a heart rate variability (HRV), a blood pressure (BP), an oxygen saturation (SpO2), an electrodermal activity (EDA), a physical motion, a breathing rate (BR), a body temperature (BT), and any combination thereof.

In many embodiments, a mobile device that is operably connected to the one or more biosensor devices wherein the instructions are executable, at least in part, by the mobile device. The system may include a networked computational server that is operably connected to the mobile device wherein the instructions are executable, at least in part, by the networked computational server.

A system having a biosensor device and a mobile device used by a user, the mobile device operably connected to the biosensor device used by the user, the system having at least one hardware processor and at least one machine-readable media for storing instructions that perform operations for health monitoring, the operations having the steps of 1) monitoring data generated by the biosensor device, the data corresponding to one or more biological properties of an individual; 2) detecting an anomaly of the data; 3 determining whether the anomaly is a false positive anomaly or a true positive anomaly by querying the user and/or analyzing the data; and 4) training one or more machine learning (ML) models on the anomaly. In this embodiment, the one or more biological properties includes a biological property selected from a group consisting of: a heart rate (HR), a heart rate variability (HRV), a blood pressure (BP), an oxygen saturation (SpO2), an electrodermal activity (EDA), a physical motion, a breathing rate (BR), a body temperature (BT), and any combination thereof. In this embodiment, the instructions have one or more neural networks comprising an autoencoder functionality tuned to have high recall, and the autoencoder configured to perform the monitoring of the data and the detecting of the anomaly of the data. Further, the one or more neural networks may include a false positive reduction (FPR) functionality configured to perform the determining whether the anomaly is the false positive anomaly or the true positive anomaly. Further, the one or more neural networks may include a classification engine functionality configured to perform a classifying of the anomaly and to perform a reporting of the anomaly to a pre-determined logic of the instructions.

A system having a biosensor device and a mobile device used by a user, the mobile device operably connected to the biosensor device used by the user, the system including at least one hardware processor and at least one machine-readable media for storing instructions that perform operations for health monitoring, the instructions having one or more neural networks comprising an autoencoder functionality tuned to have high recall, and the autoencoder configured to perform the monitoring of the data and the detecting of the anomaly of the data, the operations having the steps of 1) monitoring data generated by the biosensor device, the data corresponding to one or more biological properties of an individual, wherein the one or more biological properties includes a biological property selected from a group consisting of: a heart rate (HR), a heart rate variability (HRV), a blood pressure (BP), an oxygen saturation (SpO2), an electrodermal activity (EDA), a physical motion, a breathing rate (BR), a body temperature (BT), and any combination thereof; 2) detecting an anomaly of the data; 3) determining whether the anomaly is a false positive anomaly or a true positive anomaly by querying the user and/or analyzing the data; and 4) training one or more machine learning (ML) models on the anomaly. In this embodiment, the one or more neural networks may include a false positive reduction (FPR) functionality configured to perform the determining whether the anomaly is the false positive anomaly or the true positive anomaly.

In one embodiment, the system provides a system for health monitoring, comprising essentially of one or more hardware processors and one or more machine readable media storing instructions thereon. The instructions, executable by at least one hardware processor, cause the hardware processor to perform operations for health monitoring and management of one or more health conditions, diseases, or disorders. The operations include monitoring data which corresponds to one or more biological properties of the user, detecting an anomaly of the data, and determining whether the anomaly is a false positive anomaly or a true positive anomaly.

The system may be utilized in a method of monitoring an user's health that comprises the operations as performed by the one or more hardware processors. In some embodiments, the method may be performed by one or more individuals, including the individual monitored by the system, another individual such as a healthcare provider, or both. In this manner, the method may be performed by the system, optionally combined with one or more users or individuals.

The operations of the method comprise monitoring data which corresponds to one or more biological properties of the individual, detecting one or more anomalies within the data, and determining whether the one or more anomalies is a false positive anomaly or a true positive anomaly. In embodiments, the method is performed by the biological signal (biosignal) anomaly detection AI engine, which may be comprised of one or more neural networks or a subset of neural networks thereof.

Generally, one or more neural networks comprises an autoencoder functionality, which may be a neural network or a subset thereof, tuned to have high recall and configured to perform the evaluating of the data and the detecting of one or more anomalies of the data. An autoencoder is commonly known as a type of artificial neural network used to learn efficient codings of unlabeled data (unsupervised learning). Because the autoencoder functionality has a high recall rate, it may not reject as many potentially positive anomalies as other approaches. This may result in a larger percentage of false positive anomalies at this stage of the process than may occur with a higher detection threshold, however, one advantage of the present invention may include the use of more than one neural network for identifying true positive anomalies and removing false positive anomalies.

As such, the one or more neural networks may also comprise a false positive reduction (FPR) functionality, which may be a neural network or a subset thereof, configured to perform the determining whether the anomaly is a false positive anomaly or a true positive anomaly. The initial anomaly detection may have an intentionally lower threshold for detection and the resulting higher number of false positive anomalies may be filtered out at the FPR stage.

In some embodiments, the system further comprises a classification engine functionality configured to classify the anomaly and to report the anomaly to a pre-determined logic of the system for further processing. The classification engine functionality may be a pre-determined logic or may be a neural network such as an artificial neural network. The classification engine functionality classifies the anomaly and, optionally, engages the automated conversational agent to query the individual to assist the FPR functionality with the determination of whether the anomaly is a true positive anomaly or a false positive anomaly.

In some embodiments, the method further comprises training one or more machine learning (ML) models on the anomaly. The one or more ML models may be initialized with population or group data, then be trained to data containing any frequency of anomalies, as this frequency may differ based on the individual, the individual's health condition(s), the familiarity of the system with the individual, and other variables. Accordingly, in some embodiments the one or more ML models may be initialized using a stub, a minimal, or a placeholder data set as a starting point.

In some embodiments, the determining whether the anomaly is the false positive anomaly or the true positive anomaly comprises querying the individual, analyzing the data, or both. In embodiments in which the individual is queried, this may be performed using the automated conversational agent to improve the one or more models of the system. In embodiments in which the data is analyzed, this may be accomplished by using the existing one or more models of the system to evaluate the anomaly in view of the biological signals captured at the time of the anomaly, optionally in view of any secondary considerations such as environmental effects on the individual.

Exemplary biological properties that may be monitored by the method include a heart rate (HR), a heart rate variability (HRV), a blood pressure (BP), an oxygen saturation (SpO2), an electrodermal activity (EDA), a physical motion, a breathing rate (BR), a body temperature (BT), a blood sugar, a perspiration level, or a body metric and any combination thereof. The body metric may be any data regarding a user's measurable biological properties not specifically enumerate herein or not yet contemplated capable of measuring any measurable data about a biological property of a user or other being. Appropriate biosensors may be employed for a particular embodiment. In this manner, the number and type of biosensors may vary according to need.

In other embodiments, the one or more devices comprises one or more biosensor devices (e.g., one or more wearable biosensor devices that may be worn at any position on the body such as, but not limited to, a wrist, finger, head, face, chest, leg, ankle . . . etc. attachedable by strap, adhesive, sticker, clothing . . . etc.), and the one or more biosensor devices is configured to generate the data. In embodiments, the one or more devices comprises a mobile device which is operably connected to the one or more biosensor devices, wherein the one or more logics is executable, at least in part, by the mobile device. In such embodiments, the mobile device may be configured to execute all or part of the one or more logics of the system. In some embodiments, the one or more devices comprises a networked server which is operably connected to the one or more biosensor devices, or the mobile device, such that the one or more logics is executable, at least in part, by the networked server. In this manner, the networked server may provide all or part of the necessary computational power for performing the method of the invention. However, in embodiments, a substantial portion of the computation is performed by the mobile device or the one or more biosensor devices. In embodiments in which the one or more biosensor devices both generates the data and performs all or part of the method, the system may operate without a mobile device such as a smartphone, and without the networked server.

The system of the present system leverages several significant innovations driven by artificial intelligence (AI) engines to solve the growing market need for remote health monitoring. These innovations include multimodal objective biometrics from wearable technology, AI for detection and confirmation of anomalies, and in embodiments, the seamless integration of AI workflows with human clinician experts.

Another object of the present system is to provide a system and method that may be readily employed to benefit at-risk populations and scale a strained healthcare system to benefit those most in need.

Other objects, features, and advantages of the present system will become apparent from the following detailed description taken in conjunction with the accompanying drawings.

### BRIEF DESCRIPTION OF THE DRAWINGS

The embodiments set forth in the drawings are illustrative and exemplary in nature and not intended to limit the subject matter. The following detailed description of the illustrative embodiments can be understood when read in conjunction with the following drawings, where like structure is indicated with like reference numerals and in which:

FIG. 1 depicts an exemplary biosensor device next to an exemplary HRV graph.

FIG. 2 depicts a set of exemplary biosensor devices next to an exemplary skin conductance response (SCR) graph, showing EDA as a function of time according to one or more embodiments shown and described herein;

FIG. 3 depicts a diagram of an architecture of an exemplary autoencoder neural network with a hidden layer according to one or more embodiments shown and described herein;

FIG. 4 depicts a diagram of an architecture of an exemplary autoencoder with a plurality of hidden layers according to one or more embodiments shown and described herein;

FIG. 5 depicts a diagram of an exemplary plurality of neural networks according to the present system, comprised of an autoencoder neural network and a false positive reduction (FPR) neural network according to one or more embodiments shown and described herein;

FIG. 6 depicts a flow diagram of an exemplary system of the present system, detecting and confirming anomalies and training one or more models of the system according to one or more embodiments shown and described herein; and

FIG. 7 depicts a block diagram of a machine in the example form of a computer system within which instructions may be executed to cause the machine to perform any one or more of the methodologies discussed herein according to one or more embodiments shown and described herein.

### DETAILED DESCRIPTION

Reference is made herein to the attached drawings. Like reference numerals are used throughout the drawings to depict like or similar elements of the system. The figures are intended for representative purposes only and should not be considered limiting in any respect.

Upon serious viral infection, individuals may experience fever (>37.5° C.), dry cough, fatigue, myalgia, sore throat, nasal congestion, shortness of breath (dyspnea), acute respiratory distress syndrome (ARDS) and, rarely, diarrhea, nausea, and vomiting, among others. In many scenarios, these symptoms may be ignored or masked by an individual's preexisting condition, such as allergies or a non-serious viral infection. In such cases, the individual may be seriously ill but may not be aware of it, and healthcare may be delayed or even missed altogether. The present system provides an AI-based biometrics system for continuous or discrete monitoring of suspected and confirmed cases of viral infection, bacterial infection, mental illness, and any other health condition detectable by one or more biometrics. The system is an end-to-end platform that is comprised of a biosensor device, a biological signal anomaly detection AI engine, an automated conversation agent, real-time connectivity with health care providers, and a regulatory-compliant (e.g., HIPAA-compliant) backend.

In some embodiments, the system occurs on a mobile device or user device. It is noted that the terms “mobile device” and “user device” may be used interchangeably as used and defined herein. The “mobile device” or “user device” may be any cell phone, tablet, smart phone, mobile display, other display, self-contained computer, or any other similar device whether mobile or otherwise immobile operated by a user, person, robot, or other computer. Further, the term “user” may be any person, human, animal, computer, robot, or other being (living or not). In embodiments, the biosensor device is comprised of a wrist worn sensor which accurately measures a heart rate (FIR), a heart rate variability (HRV), a blood pressure (BP), an oxygen saturation (SpO2), an electrodermal activity (EDA), a physical motion, a breathing rate (BR), a body temperature (BT), a blood sugar, a perspiration level, or any other user metric not specifically mentioned herein, and any combination thereof. Generally, the quantitative assessment of biological signals such as HRV can be used to provide insights on disease detection and progression. Accordingly, in embodiments, the present system includes an AI engine configured to accurately detect anomalous biological signal values and report them to the higher-level logic on a device, such as a companion smartphone, where appropriate action can be taken. The biosensor device may be worn by the user or may be auxiliary (e.g. wall mounted camera, external camera or infrared reader, located on the mobile device, or any other sensor used to measure and collect biometric data).

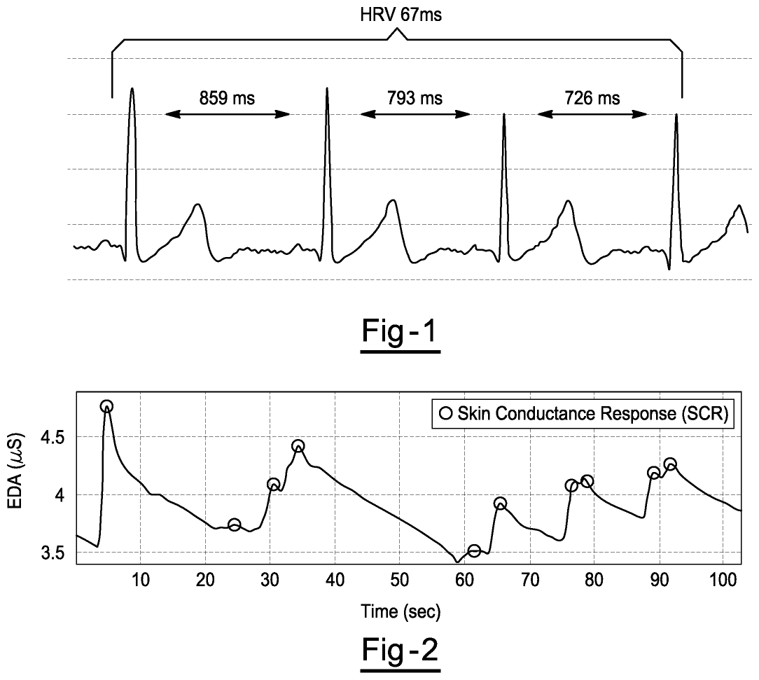

Referring now to FIG. 1 and FIG. 2, there are depicted an exemplary HRV graph (FIG. 1) and a set of exemplary biosensor devices next to an exemplary skin conductance response (SCR) graph, showing EDA as a function of time (FIG. 2). In the shown embodiment, HRV may be detected by a biosensor device configured to detect heart rate, such as a biosensor wrist band. These form factors require minimal expertise for operation and provide reasonably accurate and precise heart rate measurements for evaluation of HRV over time. Similarly, in the shown embodiment, SCR may be detected by a biosensor device configured to detect conductivity of the skin, such as a biosensor finger band. Because HRV and skin conductance may change with activation of different body systems, such as the sympathetic nervous system, the parasympathetic nervous system, the immune system, and a combination thereof, HRV and SCR may be representative of a multitude of different health conditions which require monitoring. Differentiating between these different health conditions is possible with the system and method of the present system.

In embodiments, the biosensor device (which may or may not be wearable) may be configured to measure blood pressure (BP). Because BP may be an indicator of short-term stress as well as long term illness, BP measurements may be utilized by the system of the present system to evaluate an individual. As a non-limiting example, BP measurements may be utilized to determine the risk of severe effects of a viral infection such as infection by the coronavirus which causes COVID-19, and knowing the individual's BP may affect algorithmic thresholds for anomaly detection, processing, and/or classification. In embodiments, one or more biosignals may be measured by a wearable wristband biosensor. In embodiments, the wearable wristband biosensor may be configured to measure BP.

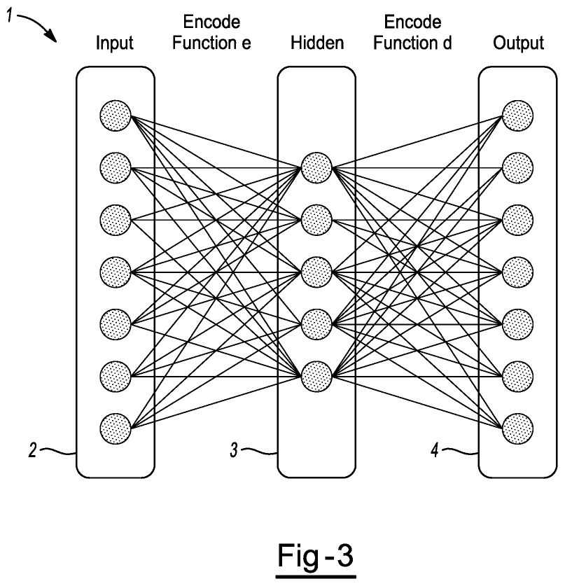

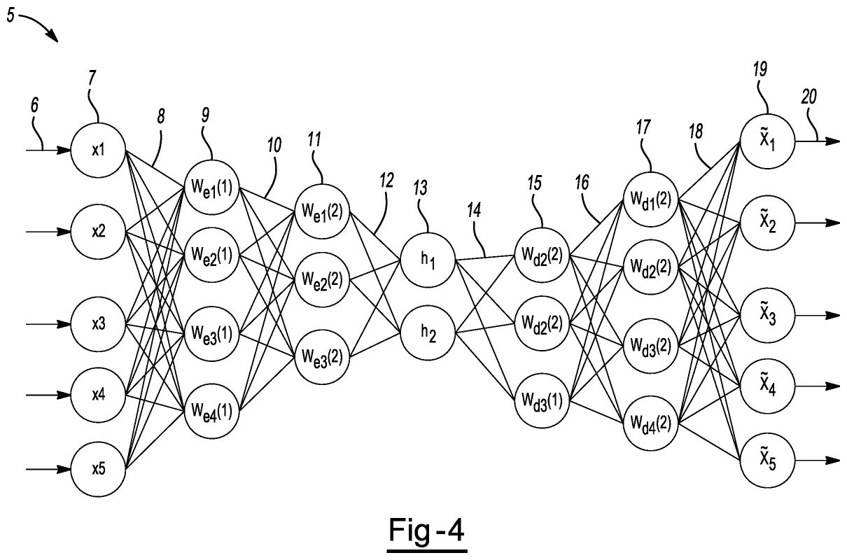

Referring now to FIG. 3 and FIG. 4, there are depicted a diagram of an architecture of an exemplary autoencoder neural network with a hidden layer (FIG. 3) and a diagram of an architecture of an exemplary autoencoder with a plurality of hidden layers (FIG. 4). A first autoencoder 1 is comprised of an input layer 2, a hidden layer 3, and an output layer 4. It may be beneficial to have the input layer 2 be larger than the hidden layer 3, because with this approach, the model is forced to create a compressed representation of the data in the one or more hidden layers by learning correlations in the data. The encoding step is comprised of an encode function, and the decoding step is comprised of a decode function. The encode-decode functions occur by multiplying an input data vector with a weight matrix, adding a bias term, and applying to the resulting vector a non-linear operation (FIG. 3). A second autoencoder 5 is comprised of a plurality of hidden layers (9, 11, 13, 15, 17) connected by edges (10, 12, 14, 16). An input layer 7 is connected to a first hidden layer 9 by edges 8 and receives data input 6. An output layer 19 is connected to a fifth hidden layer 17 by edges 18 and sends data output 20. In embodiments, the autoencoder neural network of the present system may have the same number of neurons in the output layer as the input layer, which suggests that the number of inputs and outputs may be the same. The autoencoder encodes the data into a small code (compression) and decodes it back to reproduce the input (uncompression). In embodiments, the output vector approximates the input vector, and in this manner, the autoencoder of the present system is particularly useful for unlabeled datasets.

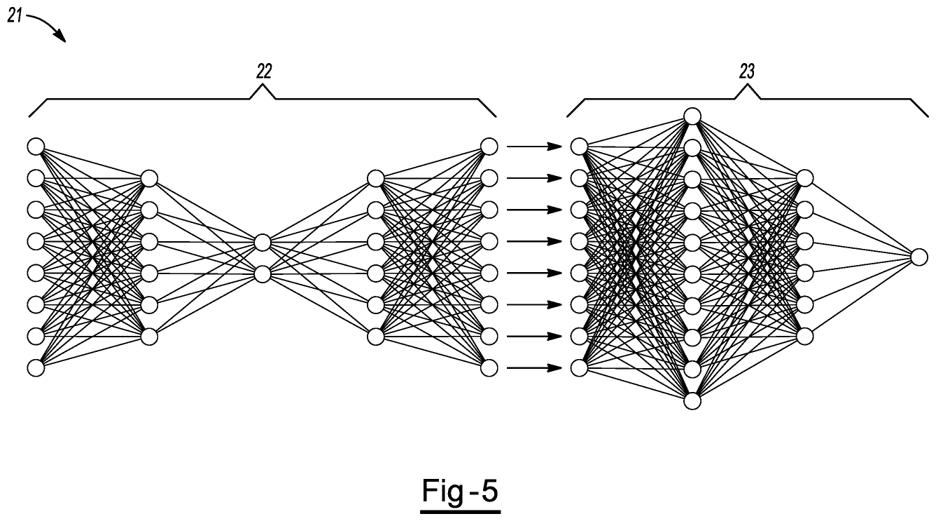

Referring now to FIG. 5, there is depicted a diagram of an exemplary plurality of neural networks according to the present system, comprised of an autoencoder neural network and a false positive reduction (FPR) neural network. In the shown embodiment, the plurality of neural networks 21 is comprised of an autoencoder neural network 22 and a FPR neural network 23. A neuron count of the input of the autoencoder neural network 22 is equal to a neuron count of the output thereof and is also equal to a neuron count of the input of the FPR neural network 23. However, a neuron count of the output of the FPR neural network 23 is less than the neuron count of the input thereof and may contribute to the ability of the FPR neural network 23 to perform its task of false positive reduction. In this manner, the plurality of neural networks 21 provides both high recall and high precision.

In embodiments, the AI engine may reside on a machine-readable media of the smartphone and be readable by one or more processors of the smartphone, such that the AI engine executes time series anomaly detection using a three-part system: an autoencoder neural network, a false positive reduction (FPR) neural network, and a classification engine functionality. In embodiments, the autoencoder uses a sliding window, from a few seconds to one-minute or more, of all sensor data to detect anomalies, but it is tuned to have high recall. This may be necessary to reduce massive amounts of data into a set of likely events. The candidate events are passed through the FPR neural network, with only the most likely being selected, due in part to processing of multiple biological signals at once (e.g., HR, HRV, skin conductance, etc.). The final step is to classify the event, if possible, so that predetermined logic can be executed. If there is any doubt of the classification, then the user or individual may be queried to identify the event. This approach allows the FPR neural network to be easily trained on tagged data, and a limited amount of verification is necessary. In embodiments, the automated conversational agent may provide tagging of anomalies, e.g., from the individual, which can facilitate training of the model(s) of the system and ensure adaptation of the system. In this manner, the system is more efficiently and effectively trained, and may be improved compared to data-intense approaches.

The autoencoder functionality may utilize a sliding window of all sensor data to detect anomalies, with high recall, to reduce massive amounts of data into a set of likely events. The FPR neural network may be comprised of a feed forward architecture, a convolutional neural network, or a recurrent neural network, according to need for a particular embodiment. The classification engine functionality may be a neural network, or it may not be a neural network, according to a particular need or embodiment. The classification engine functionality may be configured to capture multiple inputs and produce secondary data sets (e.g., combinations) that rapidly increases the complexity of the calculations. The classification engine functionality may be assisted by the automated conversation agent, with input from the individual, to classify the anomaly as needed. In embodiments, the classification engine functionality may be a support vector machine (SVM), a random forest algorithm, or a boosted decision tree algorithm, which are non-neural network algorithms for manipulating data by adding dimensional information to classify data. In embodiments, the classification engine may be any suitable classical machine learning algorithm, according to need. In embodiments, the classification engine functionality may be a random forest algorithm. In embodiments, the system may provide a time-series analysis.

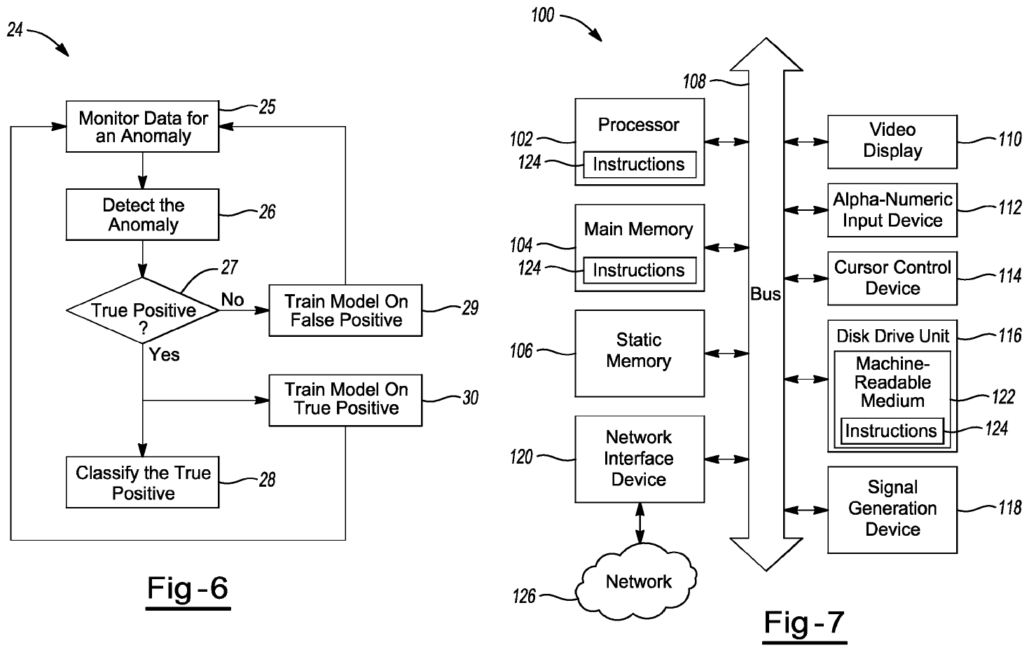

Referring now to FIG. 6, there is depicted a flow diagram of an exemplary system of the present system, detecting and confirming anomalies and training one or more models of the system. A system 24 is configured to monitor 25 data for an anomaly. When one or more anomalies is detected 26 by the autoencoder neural network, it is passed to the FPR neural network to determine 27 whether it is a true positive. If it is not a true positive, the one or more ML models is trained 29 on the false positive. If it is a true positive, the true positive is classified 28 by the classification engine functionality and the one or more ML models is trained 30 on the true positive. In this manner, the system is configured to become customized for use by the individual, and “learns” from past successes and failures.

An automated conversation agent driven by speech, text, or both, is a part of the system that can complete triage, condition assessment, and information gathering. The agent can be triggered by anomaly detection or by backend human management requests (e.g., assign user to fill out of pre-existing form). Commercial grade dialog-building systems may be utilized, as well as customized bidirectional encoder representations for transformers (BERT-based algorithm) for information extraction to fill in as much required data as the user provides, thus limiting the length of an agent interaction. In embodiments, the agent supports the three levels of biological signal anomaly detection, e.g., by allowing for bulk training on the autoencoder functionality, and faster customization of the system on the FPR and classification levels. In embodiments, the agent may be used to ask the individual questions such as, “What happened earlier?” and the individual may respond, “I coughed,” or “I have a headache,” or “I fell down,” etc. This agent allows for continuous improvement and learning of additional anomalies. Once enough samples are classified, the system will only ask what happened with anomalies that do not match existing classes. In embodiments, the system may be bootstrapped with initial training data collected in controlled conditions from many individuals.

During early training of one or more ML models of the system, frequent input from the individual using the system may be required to better fit input data structures to output data structures so as to adapt the one or more ML models to the individual. During later training of one or more ML models of the system, the input from the individual may become less frequent as the model becomes better adapted to identify and remove false positive anomalies. Instead of an operator manually removing the false positive anomalies, the one or more ML models is trained by the individual. Using output from the automated conversational agent, the system trains the one or more ML models to correlate the false positive anomalies with the biological signals obtained and thereby rely less on the automated conversational agent when these biological signals are observed thereafter.

In embodiments, the present system provides a simple, easy-to-use interface for communicating with a health care provider, with either live chat or with the AI-driven mode. This enables direct communication with parties to request additional information, answer questions, or offer therapeutic suggestions. In embodiments, the secure backend data architecture summarizes user information (plots, charts, event logs) concerning progress and outcomes to the care team. Additionally, it flags potential issues based on the results. It also allows healthcare providers to set reminders for remote users and schedule questionnaires. Access is secure, restricted, and logged. All data and logs are encrypted in flight and on the back-end database. In this manner, security and regulatory compliance are attained.

The present system provides a strong foundation that can be easily scaled to address remote healthcare monitoring during the current COVID-19 crisis, as well as future pandemics. One difference between use of the system in a pandemic versus mental health monitoring is the types and combinations of biological signals to be captured. The reference design hardware technology (e.g., as may be provided by Maxim Integrates) may be able to collect a wide array of biological signals including a heart rate (HR), a heart rate variability (HRV), a blood pressure (BP), an oxygen saturation (SpO2), an electrodermal activity (EDA), a physical motion, a breathing rate (BR), a body temperature (BT), and any combination thereof. However, scaling the platform to integrate more of these parameters we will be able to directly address symptoms of COVID-19, as well as underlying chronic disease conditions that also require these parameters. The system also leverages a secondary reference design (e.g., as may be obtained from Philips Research) that integrates electrodermal activity. Coupled with the biological signal front end hardware, the secure mechanism for cloud data transmission and storage facilitates remote data collection, with algorithms to scale and reduce data living both on a smart phone application, as well as in the cloud.

In embodiments, the actual combination of biological signals to be recorded may be determined as data in the field continues to rapidly emerge. This aligns with a major technical innovation of the system, to adaptively change the combinations and types of biological signals that can be recorded. More specifically, current research indicates early disease indicators include fever (58.8%) cough (55.7%), dyspnea (up to 50%), and fatigue (33%). Another indicator was gastrointestinal symptoms. These are all symptoms that can be remotely measured by scaling the system and method. More specifically, the system can capture fever with the temperature sensor, cough and fatigue with the motion and HRV sensors, and dyspnea with the SpO2 sensor. Furthermore, a baseline for these indicators can be established before symptoms appear. For example, a good model of a user's body temperature and level of tiredness at various times during the day may be obtained. The system can then verify the authenticity of detected symptoms (coughing, tiredness), and ask for indicators of other symptoms (like gastrointestinal issues) on a regular basis. If there is any indication of possible COVID-19, we can put them in touch with frontline telehealth providers.

In order to prove the efficacy of the technology, data may be collected from large and targeted cohorts of people who fall into four groups: (1) healthy controls and (2) those who have COVID-19 like symptoms, including cold, pneumonia, fever, fatigue, coughing, and gastrointestinal issues, but receive a negative diagnosis, and 3) those who have COVID-19 like symptoms, including cold, pneumonia, fever, fatigue, coughing, and gastrointestinal issues and receive a positive diagnosis, and 4) those who are asymptomatic, but received a positive diagnosis. This data may be used to develop and validate targeted algorithms for disease detection and monitoring progression.

In embodiments, the system of the present system may be utilized continuously, such that the method is constantly or nearly-constantly performed to continuously monitor the individual. Advantages of continuous monitoring include the detection of subtle symptoms of a condition, disease, or disorder, and the opportunity for the system to adapt to the individual to improve sensitivity and accuracy. In this manner, the system may be reliably and continuously used by the individual.

In embodiments, the system may be discretely used, such that use of the system is not continuous. By using the system discretely to obtain snapshots of the individual's health throughout an observation period, the number of candidate events observed may be much lower, and the data processing requirements of the system may be altered as a result. In addition, if the biosensor device is not worn continuously, discrete monitoring of the individual enables the system to evaluate the individual using only periods in which the individual wishes to be monitored. In this manner, the individual may control the degree to which the system is utilized in a particular embodiment.

The operations, algorithms, and methods of the present system may generally be implemented in suitable combinations of software, hardware, firmware, or a combination thereof, and the provided functionality may be grouped into a number of components, modules, or mechanisms. Modules can constitute either software modules (e.g., code embodied on a non-transitory machine-readable medium) or hardware-implemented modules. A hardware-implemented module is a tangible unit capable of performing certain operations and can be configured or arranged in a certain manner. In example embodiments, one or more computer systems (e.g., a standalone, client, or server computer system) or one or more processors can be configured by software (e.g., an application or application portion) as a hardware-implemented module that operates to perform certain operations as described herein.

In embodiments, a hardware-implemented module can be implemented mechanically or electronically. For example, a hardware-implemented module can comprise dedicated circuitry or logic that is permanently configured (e.g., as a special-purpose processor, such as a field programmable gate array (FPGA) or an application-specific integrated circuit (ASIC)) to perform certain operations. A hardware-implemented module can also comprise programmable logic or circuitry (e.g., as encompassed within a general-purpose processor or other programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) can be driven by cost and time considerations.

Accordingly, the term “hardware-implemented module” should be understood to encompass a tangible entity, be that an entity that is physically constructed, permanently configured (e.g., hardwired), or temporarily or transitorily configured (e.g., programmed) to operate in a certain manner, to perform certain operations described herein, or both. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor can be configured as respective different hardware-implemented modules at different times. Software can accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware-implemented modules can be regarded as being communicatively coupled. Where multiple such hardware-implemented modules exist contemporaneously, communications can be achieved through signal transmission (e.g., over appropriate circuits and buses that connect the hardware-implemented modules). In embodiments in which multiple hardware-implemented modules are configured or instantiated at different times, communications between such hardware-implemented modules can be achieved, for example, through the storage and retrieval of information in memory structures to which the multiple hardware-implemented modules have access. For example, one hardware-implemented module can perform an operation and store the output of that operation in a memory device to which it is communicatively coupled. A further hardware-implemented module can then, at a later time, access the memory device to retrieve and process the stored output. Hardware-implemented modules can also initiate communications with input or output devices, and can operate on a resource (e.g., a collection of information).

The various operations of example methods described herein can be performed, at least partially, by one or more processors that are temporarily configured (e.g., by software) or permanently configured to perform the relevant operations. Whether temporarily or permanently configured, such processors can constitute processor-implemented modules that operate to perform one or more operations or functions. The modules referred to herein can, in some example embodiments, comprise processor-implemented modules.

Similarly, the methods described herein can be at least partially processor-implemented. For example, at least some of the operations of a method can be performed by one of processors or processor-implemented modules. The performance of certain of the operations can be distributed among the one or more processors, not only residing within a single machine, but deployed across a number of machines. In embodiments, the processor or processors can be located in a single location (e.g., within an office environment, or a server farm), while in other embodiments the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), these operations being accessible via a network (e.g., the Internet) and via one or more appropriate interfaces (e.g., application program interfaces (APIs)).

Example embodiments can be implemented in digital electronic circuitry, in computer hardware, firmware, or software, or in combinations thereof. Example embodiments can be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of description language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

In example embodiments, operations can be performed by one or more programmable processors executing a computer program to perform functions by operating on input data and generating output. Method operations can also be performed by, and apparatus of example embodiments can be implemented as, special purpose logic circuitry, e.g., an FPGA or an ASIC.

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine) and software architectures that can be deployed, in various example embodiments.

FIG. 7 is a block diagram of a machine in the example form of a computer system 100 within which instructions 124 may be executed to cause the machine to perform any one or more of the methodologies discussed herein. In alternative embodiments, the machine operates as a standalone device or can be connected (e.g., networked) to other machines. In a networked deployment, the machine can operate in the capacity of a server or a client machine in server-client network environment, or as a peer machine in a peer-to-peer (or distributed) network environment. The machine can be a personal computer (PC), a tablet PC, a set-top box (STB), a personal digital assistant (PDA), a cellular telephone, a web appliance, a network router, switch, or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 100 includes a processor 102 (e.g., a central processing unit (CPU), a graphics processing unit (GPU), or both), a main memory 104, and a static memory 106, which communicate with each other via a bus 108. The computer system 100 can further include a video display 110 (e.g., a liquid crystal display (LCD) or a cathode ray tube (CRT)). The computer system 100 also includes an alpha-numeric input device 112 (e.g., a keyboard or a touch-sensitive display screen), a user interface (UI) navigation (or cursor control) device 114 (e.g., a mouse), a disk drive unit 116, a signal generation device 118 (e.g., a speaker), and a network interface device 120.

The disk drive unit 116 includes a machine-readable medium 122 on which are stored one or more sets of data structures and instructions 124 (e.g., software) embodying or utilized by any one or more of the methodologies or functions described herein. The instructions 124 can also reside, completely or at least partially, within the main memory 104 or within the processor 102, or both, during execution thereof by the computer system 100, with the main memory 104 and the processor 102 also constituting machine-readable media.

While the machine-readable medium 122 is shown in an example embodiment to be a single medium, the term “machine-readable medium” can include a single medium or multiple media (e.g., a centralized or distributed database, and/or associated caches and servers) that store the one or more instructions 124 or data structures. The term “machine-readable medium” shall also be taken to include any tangible medium that is capable of storing, encoding, or carrying instructions 124 for execution by the machine and that cause the machine to perform any one or more of the methodologies of the present disclosure, or that is capable of storing, encoding, or carrying data structures utilized by or associated with such instructions 124. The term “machine-readable medium” shall accordingly be taken to include, but not be limited to, solid-state memories, and optical and magnetic media. Specific examples of machine-readable media 122 include non-volatile memory, including by way of example semiconductor memory devices, e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks.

The instructions 124 can be transmitted or received over a communication network 126 using a transmission medium. The instructions 124 can be transmitted using the network interface device 120 and any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi and WiMax networks). The term “transmission medium” shall be taken to include any intangible medium that is capable of storing, encoding, or carrying instructions 124 for execution by the machine, and includes digital or analog communications signals or other intangible media to facilitate communication of such software.

The foregoing descriptions of specific embodiments of the present system have been presented for purposes of illustration and description. They are not intended to be exhaustive or to limit the present system to the precise forms disclosed, and modifications and variations are possible in view of the above teaching. The exemplary embodiment was chosen and described to best explain the principles of the present system and its practical application, to thereby enable others skilled in the art to best utilize the present system and its embodiments with modifications as suited to the use contemplated.

With respect to the description provided herein, it is submitted that the optimal features of the system include variations in size, materials, shape, form, function and manner of operation, assembly, and use. All structures, functions, and relationships equivalent or essentially equivalent to those disclosed are intended to be encompassed by the present system. It is noted that the terms “substantially” and “about” may be utilized herein to represent the inherent degree of uncertainty that may be attributed to any quantitative comparison, value, measurement, or other representation. Any values that may be modified by such terminology are also part of the teachings herein. For example, if a teaching recited “about 10,” the skilled person should recognize that the value of 10 is also contemplated.

These terms are also utilized herein to represent the degree by which a quantitative representation may vary from a stated reference without resulting in a change in the basic function of the subject matter at issue.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

While particular embodiments have been illustrated and described herein, it should be understood that various other changes and modifications may be made without departing from the spirit and scope of the claimed subject matter.

Moreover, although various aspects of the claimed subject matter have been described herein, such aspects need not be utilized in combination.

It is therefore intended that the appended claims (and/or any future claims filed in any corresponding application) cover all such changes and modifications that are within the scope of the claimed subject matter.

I claim:

1) A system having a biosensor device used by a user, the system comprising:
at least one hardware processor; and
at least one machine-readable media for storing instructions that cause the at least one hardware processor to perform operations for health monitoring when executed by the one or more hardware processors, the operations comprising the steps of:
monitoring data generated by the biosensor device, the data corresponding to one or more biological properties of an individual;
detecting an anomaly of the data; and
determining whether the anomaly is a false positive anomaly or a true positive anomaly by querying the user and/or analyzing the data.

2) The system of claim 1, wherein the instructions have one or more neural networks comprising an autoencoder functionality tuned to have high recall, and the autoencoder configured to perform the monitoring of the data and the detecting of the anomaly of the data.

3) The system of claim 2, wherein the one or more neural networks comprises a false positive reduction (FPR) functionality configured to perform the determining whether the anomaly is the false positive anomaly or the true positive anomaly.

4) The system of claim 3, wherein the one or more neural networks comprises a classification engine functionality configured to perform a classifying of the anomaly and to perform a reporting of the anomaly to a pre-determined logic of the instructions.

5) The system of claim 1, wherein the operations further comprise training one or more machine learning (ML) models on the anomaly.

6) The system of claim 1, wherein the one or more biological properties comprises a biological property selected from a group consisting of: a heart rate (HR), a heart rate variability (HRV), a blood pressure (BP), an oxygen saturation (SpO2), an electrodermal activity (EDA), a physical motion, a breathing rate (BR), a body temperature (BT), a blood sugar level, a perspiration level, or a body metric, and any combination thereof.

7) The system of claim 1, wherein the system comprises a user device that is operably connected to the one or more biosensor devices;
wherein the instructions are executable, at least in part, by the user device.

8) The system of claim 8, wherein the system comprises a networked computational server that is operably connected to the user device;
wherein the instructions are executable, at least in part, by the networked computational server.

9) A system having a biosensor device and a user device used by a user, the user device operably connected to the biosensor device used by the user, the system comprising:
at least one hardware processor; and
at least one machine-readable media for storing instructions that perform operations for health monitoring, the operations comprising the steps of:
monitoring data generated by the biosensor device, the data corresponding to one or more biological properties of an individual;
detecting an anomaly of the data;
determining whether the anomaly is a false positive anomaly or a true positive anomaly by querying the user and/or analyzing the data; and
training one or more machine learning (ML) models on the anomaly.

10) The system of claim 9, wherein the one or more biological properties comprises a biological property selected from a group consisting of: a heart rate (HR), a heart rate variability (HRV), a blood pressure (BP), an oxygen saturation (SpO2), an electrodermal activity (EDA), a physical motion, a breathing rate (BR), a body temperature (BT), a body metric, and any combination thereof.

11) The system of claim 9, wherein the instructions have one or more neural networks comprising an autoencoder functionality tuned to have high recall, and the autoencoder configured to perform the monitoring of the data and the detecting of the anomaly of the data.

12) The system of claim 11, wherein the one or more neural networks comprises a false positive reduction (FPR) functionality configured to perform the determining whether the anomaly is the false positive anomaly or the true positive anomaly.

13) The system of claim 12, wherein the one or more neural networks comprises a classification engine functionality configured to perform a classifying of the anomaly and to perform a reporting of the anomaly to a pre-determined logic of the instructions.

14) The system of claim 9, wherein the hardware processor is integrated within the user device.

15) The system of claim 9, wherein the biosensor device is integrated within the user device.

16) The system of claim 9, wherein the user device is self-contained including both the biosensor device and the hardware processor on the user device.

17) A system having a biosensor device and a user device used by a user, the user device operably connected to the biosensor device used by the user, the system comprising:
at least one hardware processor; and
at least one machine-readable media for storing instructions that perform operations for health monitoring, the instructions having one or more neural networks comprising an autoencoder functionality tuned to have high recall, and the autoencoder configured to perform the monitoring of the data and the detecting of the anomaly of the data, the operations comprising the steps of:
monitoring data generated by the biosensor device, the data corresponding to one or more biological properties of an individual, wherein the one or more biological properties comprises a biological property selected from a group consisting of: a heart rate (HR), a heart rate variability (HRV), a blood pressure (BP), an oxygen saturation (SpO2), an electrodermal activity (EDA), a physical motion, a breathing rate (BR), a body temperature (BT), a blood sugar level, a perspiration level, or a body metric and any combination thereof;
detecting an anomaly of the data;
determining whether the anomaly is a false positive anomaly or a true positive anomaly by querying the user and/or analyzing the data; and
training one or more machine learning (ML) models on the anomaly.

18) The system of claim 17, wherein the one or more neural networks comprises a false positive reduction (FPR) functionality configured to perform the determining whether the anomaly is the false positive anomaly or the true positive anomaly.

19) The system of claim 17, wherein the hardware processor is integrated within the user device.

20) The system of claim 17, wherein the biosensor device is integrated within the user device.
